(19) United States
(12) Patent Application Publication
(10) Pub. No.: US 2026/0248847 A1
(43) Pub. Date: Aug. 27, 2026

(54) REDOX-MODULATING ORGANELLE COMPLEXES

(71) Applicant: LUCA Science Inc., Tokyo (JP)

(72) Inventors: Keiichi Sakakibara, Hokkaido, Sapporo (JP); Rick C. Tsai, Tokyo (JP); Masashi Suganuma, Aichi, Toyota (JP); Hisashi Ohta, Ibaraki, Tsukuba (JP)

(21) Appl. No.: 18/992,156

(22) PCT Filed: Jul. 6, 2023
(86) PCT No.: PCT/US2023/027024

§ 371 Date: Jan. 7, 2025

Related U.S. Application Data
(60) Provisional application No. 63/359,108, filed on Jul. 7, 2022.

(51) Int. Cl. A61K 35/12; A61P 39/06

(52) U.S. Cl. CPC A61K 35/12; A61P 39/06

(57) ABSTRACT

Disclosed herein include methods, compositions, and kits suitable for use in reducing or preventing oxidative stress and/or reductive stress. In some embodiments, the method comprises contacting a redox-sensitive composition with an effective amount of a disclosed redox-modulating composition. In some embodiments, the method comprises administering to a subject in need an effective amount of a disclosed redox-modulating composition. In some embodiments, the redox-modulating composition comprises isolated organelle complexes. Organelle complexes can comprise mitochondria and one or more of endoplasmic reticulum, peroxisomes, lysosomes, and Golgi apparatus. In some embodiments, the redox-modulating composition comprises isolated mitochondria. The redox-modulating composition can be capable of reducing levels of ROS and/or RNS in a redox-sensitive composition and/or cells.

### RELATED APPLICATIONS

This application claims the benefit under 35 U.S.C. § 119(e) of U.S. Provisional Patent Application Ser. No. 63/359,108, filed Jul. 7, 2022, the content of this related application is incorporated herein by reference in its entirety for all purposes.

### BACKGROUND

### Field

The present disclosure relates generally to methods of reducing and preventing oxidative stress and/or reductive stress.

### Description of the Related Art

Mitochondria are intracellular organelles responsible for a number of metabolic transformations and regulatory functions. Mitochondria are highly dynamic organelles that move throughout the cell and undergo structural transitions, changing the length, morphology, shape and size. Moreover, mitochondria are continuously eliminated and regenerated in a process known as mitochondrial biogenesis. While most mitochondrial genes have been transferred to the nuclear genome, the mitochondria genome still encodes rRNAs, tRNAs, and 13 subunits of the electron transport chain (ETC). Functional communication between the nuclear and mitochondrial genomes is therefore essential for mitochondrial biogenesis, efficient oxidative phosphorylation, and normal health. Mitochondria are also the major source of free radicals and reactive oxygen species (ROS) that cause oxidative stress. Additionally, mitochondria play key roles in intracellular signaling as well as control of cell death, including apoptosis and necrosis. There is growing evidence that mitochondrial dysfunction is associated with a broad range of human diseases. Mitochondrial dysfunction, for example, respiratory chain complex dysfunction, is a major cause responsible for a mitochondrial disease and aging.

Oxidative stress is caused by disturbances to the normal redox state within cells. An imbalance between routine production and detoxification of reactive oxygen species such as peroxides and free radicals can result in oxidative damage to the cellular structure and machinery. The most important source of reactive oxygen species under normal conditions in aerobic organisms is the leakage of activated oxygen from mitochondria during normal oxidative respiration. Impairments associated with this process are suspected to contribute to mitochondrial disease, neurodegenerative disease, and diseases of aging.

Reductive stress can occur in response to conditions that shift the redox balance of important biological redox couples, such as the NAD⁺/NADH, NADP⁺/NADPH, and GSH/GSSG, to a more reducing state. Overexpression of antioxidant enzymatic systems can lead to excess reducing equivalents that can deplete reactive oxidative species, driving the cells to reductive stress. Feedback regulation can be established in which chronic reductive stress induces oxidative stress, which in turn, stimulates again reductive stress. Excess reducing equivalents can regulate cellular signaling pathways, modify transcriptional activity, induce alterations in the formation of disulfide bonds in proteins, reduce mitochondrial function, decrease cellular metabolism, and thus, contribute to the development of redox diseases and disorders, such as, for example, cardiomyopathy, pulmonary hypertension, stent stenosis, muscular dystrophy, neurological disorders, Parkinson's disease, Alzheimer's disease, metabolic syndrome and insulin resistance, rheumatoid arthritis, renal diseases, and cancer.

There is a need for redox-modulating compositions and methods capable of reducing or preventing oxidative stress and/or reductive stress.

### SUMMARY

Disclosed herein include methods of reducing or preventing oxidative stress and/or reductive stress in a redox-sensitive composition. In some embodiments, the method comprises: contacting a redox-sensitive composition with an effective amount of a redox-modulating composition, thereby reducing or preventing oxidative stress and/or reductive stress in the redox-sensitive composition. In some embodiments, the redox-modulating composition comprises isolated organelle complexes. In some embodiments, the organelle complexes comprise mitochondria and one or more of endoplasmic reticulum, peroxisomes, lysosomes, and Golgi apparatus. In some embodiments, the redox-modulating composition comprises isolated mitochondria. In some embodiments, the redox-modulating composition comprises mitochondria isolated from intact cells and/or floating cells or frozen cells or combination thereof.

Disclosed herein include methods of reducing levels of reactive oxygen species (ROS) and/or reactive nitrogen species (RNS) in a redox-sensitive composition. In some embodiments, the method comprises: contacting a redox-sensitive composition with an effective amount of a redox-modulating composition, thereby reducing levels of ROS and/or RNS in the redox-sensitive composition. In some embodiments, reducing levels of ROS and/or RNS in the redox-sensitive composition thereby reduces or prevents oxidative stress and/or reductive stress in the redox-sensitive composition. In some embodiments, the redox-modulating composition comprises isolated organelle complexes. In some embodiments, the organelle complexes comprise mitochondria and one or more of endoplasmic reticulum, peroxisomes, lysosomes, and Golgi apparatus. In some embodiments, the redox-modulating composition comprises isolated mitochondria. In some embodiments, the redox-modulating composition comprises mitochondria isolated from intact cells and/or floating cells or frozen cells or combination thereof.

In some embodiments, the contacting step comprises incubating a mixture of the redox-sensitive composition and the redox-modulating composition for less than about 30 seconds. The redox-sensitive composition can be an oxidant-sensitive composition, a reductant-sensitive composition, or a combination thereof. In some embodiments, the effective amount comprises at least about 5 ug/mL to about 5 mg/mL of the redox-modulating composition. In some embodiments, the redox-sensitive composition is experiencing oxidative stress and/or reductive stress or is at risk of experiencing oxidative stress and/or reductive stress. In some embodiments, the redox-sensitive composition comprises one or more cells. The contacting step can comprise introducing the redox-modulating composition into the one or more cells. The one or more cells can be cells of a subject. The one or more cells can be undergoing or at risk of undergoing hypoxia. In some embodiments, the contacting is performed ex vivo, in vitro, or in vivo.

In some embodiments, the redox-sensitive composition comprises a biological sample. In some embodiments, the biological sample is selected from the group consisting of a soil sample, an air sample, an environmental sample, a cell culture sample, a bone marrow sample, a rainfall sample, a fallout sample, a space sample, an extraterrestrial sample, a sewage sample, a ground water sample, an abrasion sample, an archaeological sample, a food sample, a blood sample, a serum sample, a plasma sample, a urine sample, a stool sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumor sample, a tissue sample, a cell sample, a cell lysate sample, a virus culture sample, a forensic sample, an infection sample, a nosocomial infection sample, a production sample, a drug preparation sample, a biological molecule production sample, a protein preparation sample, a lipid preparation sample, a carbohydrate preparation sample, a solution of a nucleotide, a solution of polynucleotide, a solution of a nucleic acid, a solution of a peptide, a solution of a polypeptide, a solution of an amino acid, a solution of a protein, a solution of a synthetic polymer, a solution of a biochemical composition, a solution of an organic chemical composition, a solution of an inorganic chemical composition, a solution of a lipid, a solution of a carbohydrate, a solution of a combinatory chemistry product, a solution of a drug candidate molecule, a solution of a drug molecule, a solution of a drug metabolite, a suspension of a cell, a suspension of a virus, a suspension of a microorganism, a suspension of a metal, a suspension of metal alloy, a solution of a metal ion, and any combination thereof.

Disclosed herein include methods of reducing or preventing oxidative stress and/or reductive stress in a subject. In some embodiments, the method comprises: administering to the subject an effective amount of a redox-modulating composition, thereby reducing or preventing oxidative stress and/or reductive stress in the subject. In some embodiments, the redox-modulating composition comprises isolated organelle complexes. In some embodiments, the organelle complexes comprise mitochondria and one or more of endoplasmic reticulum, peroxisomes, lysosomes, and Golgi apparatus. In some embodiments, the redox-modulating composition comprises isolated mitochondria. In some embodiments, the redox-modulating composition comprises mitochondria isolated from intact cells and/or floating cells or frozen cells or combination thereof.

Disclosed herein include methods of treating or preventing a redox disease or disorder in a subject. In some embodiments, the method comprises: administering to the subject an effective amount of a redox-modulating composition, thereby treating or preventing a redox disease or disorder in a subject. In some embodiments, the redox-modulating composition comprises isolated organelle complexes. In some embodiments, the organelle complexes comprise mitochondria and one or more of endoplasmic reticulum, peroxisomes, lysosomes, and Golgi apparatus. In some embodiments, the redox-modulating composition comprises isolated mitochondria. In some embodiments, the redox-modulating composition comprises mitochondria isolated from intact cells and/or floating cells or frozen cells or combination thereof.

In some embodiments, the redox-modulating composition comprises homogenized mitochondria, first organelle complexes, and/or second organelle complexes. In some embodiments, the first organelle complexes and second organelle complexes comprise mitochondria and one or more of endoplasmic reticulum, peroxisomes, lysosomes, and Golgi apparatus. In some embodiments, the first organelle complexes and second organelle complexes are depleted of cytosolic macromolecules. In some embodiments, first organelle complexes are derived from (i) frozen cells; (ii) floating cells; and/or (iii) cells contacted with a surfactant at a concentration at or above the critical micellar concentration (CMC) for the surfactant. In some embodiments, second organelle complexes are derived from (i) adherent cells; and/or (ii) cells contacted with a surfactant at a concentration below the critical micellar concentration (CMC) for the surfactant. In some embodiments, the first organelle complexes and second organelle complexes are derived from cells treated with a mitochondria-activating agent.

In some embodiments, the homogenized mitochondria, first organelle complexes, and/or second organelle complexes are encapsulated in lipid membrane-based vesicles. In some embodiments, the effective amount comprises at least about 1 ug to about 1 mg of the redox-modulating composition. In some embodiments, the redox-modulating composition does not comprise intact cells.

In some embodiments, the oxidative stress and/or reductive stress comprises elevated levels of ROS, RNS, and/or free radicals. In some embodiments, the oxidative stress and/or reductive stress comprises altered cell functions. In some embodiments, the oxidative stress and/or reductive stress is associated with a redox disease or disorder. In some embodiments, the redox-modulating composition reduces levels of one or more ROS and/or one or more RNS in the subject or the redox-sensitive composition by at least about 5%. In some embodiments, the redox-modulating composition reduces level of oxidative cell stress and thereby recover or restore the cell functions. In some embodiments, the redox-modulating composition has a ROS scavenging activity and/or RNS scavenging activity. In some embodiments, the redox-modulating composition reduces or prevents ROS generation and/or RNS generation in the subject. In some embodiments, the redox-modulating composition has superoxide dismutase activity, catalase activity, peroxidase activity, or any combination thereof.

In some embodiments, the redox-modulating composition increases and/or decreases one or more of the following ratios in the subject or the redox-sensitive composition: oxidized to reduced forms of nicotinamide adenine dinucleotide (NAD⁺/NADH), oxidized to reduced forms of nicotinamide adenine dinucleotide phosphate (NADP⁺/NADPH), oxidized to reduced forms of glutathione (GSSG/GSH), and oxidized to reduced forms of thioredoxin (TrxSS/TrxSH₂). In some embodiments of the compositions and methods provided herein, the disclosed redox-modulating composition increases (by least about 1.1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) one or more of the following ratios in the subject or the redox-sensitive composition: oxidized to reduced forms of nicotinamide adenine dinucleotide (NAD⁺/NADH), oxidized to reduced forms of nicotinamide adenine dinucleotide phosphate (NADP⁺/NADPH), oxidized to reduced forms of glutathione (GSSG/GSH), and oxidized to reduced forms of thioredoxin (TrxSS/TrxSH₂).

In some embodiments, the redox-modulating composition has free radical scavenging activity and/or inhibits free radical formation. In some embodiments, the subject is experiencing oxidative stress and/or reductive stress or is at risk of experiencing oxidative stress and/or reductive stress (e.g., oxidative stress and/or reductive stress caused by ischemic-reperfusion injury or the redox disease or disorder).

In some embodiments, the reactive nitrogen species comprise nitric oxide (NO), nitrogen dioxide radical (·NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) or dinitrogen trioxide (N₂O₃), or any combination thereof. In some embodiments, the reactive oxygen species comprise superoxide (O₂·⁻), hydroperoxy (HO·₂), hydrogen peroxide (H₂O₂), peroxynitrite (ONOO—), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof. In some embodiments, the ROS are derived from neutrophils and/or xanthine oxidase (XO).

In some embodiments, the method comprises: identifying a subject experiencing oxidative stress and/or reductive stress or at risk of experiencing oxidative stress and/or reductive stress. In some embodiments, the method comprises: measuring the levels of RNS in the subject or the redox-sensitive composition with a probe that detects reactive nitrogen species; and/or measuring the levels of ROS in the subject or the redox-sensitive composition with a probe that detects reactive oxygen species. In some embodiments, the probe that detects reactive nitrogen species is diaminonaphthalene, a diaminofluorescein, a diaminorhodamine, a diaminocyanine, an NiSPY, dichlorodiaminocalcein, 1,2-diaminoanthraquinone, or DAMBO-P^{H}. In some embodiments, the probe that detects reactive oxygen species is 2′,7′-dichloro-fluorescein diacetate, dihydrorhodamine 123, 3′-(p-aminophenyl) fluorescein (API), 3′-(p-hydroxyphenyl) fluorescein (HPF), aminophenoxycalcein (APC), mitoAR, mitoHR, DPAX, DMAX, a hydrocyanine, or dihydroethidium.

In some embodiments, the subject has or is suspected of having a disease or condition in which ROS is implicated selected from the group consisting of atherosclerosis, heart disease, heart failure, hypertension, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, stroke, an ischemia-reperfusion injury, an ischemia-reperfusion injury, chronic inflammation, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack, UV damage to the skin, organ transplantation, surgery, and any combination thereof.

In some embodiments, administering the redox-modulating composition to the subject treats, reduces, or prevents ischemia-reperfusion injury of the subject. In some embodiments, the ischemic-reperfusion injury is caused by mitochondrial dysfunction, hypoxic injury, HMGB1 release or necrotic cell death. In some embodiments, the redox-modulating composition suppresses ischemic-reperfusion injury, mitochondrial dysfunction, hypoxic injury, necrotic cell death, or any combination thereof. In some embodiments, the redox disease or disorder comprises ROS-mediated and/or RNS-mediated oxidative damage to one or more tissues of the subject. In some embodiments, the administering comprises intravenous administration, intra-arterial administration, intra-tracheal administration, subcutaneous, administration intramuscular administration, inhalation, intrapulmonary administration, and/or intraocular administration.

In some embodiments, the redox disease or disorder is selected from the group consisting of: a mitochondrial disorder; an inherited mitochondrial disease; Alpers Disease; Barth syndrome; a Beta-oxidation Defect; Carnitine-Acyl-Carnitine Deficiency; Carnitine Deficiency; a Creatine Deficiency Syndrome; Co-Enzyme Q10 Deficiency; Complex I Deficiency; Complex II Deficiency; Complex III Deficiency; Complex IV Deficiency; Complex V Deficiency; COX Deficiency; chronic progressive external ophthalmoplegia (CPEO); CPT I Deficiency; CPT II deficiency; Friedreich's Ataxia (FA); Glutaric Aciduria Type II; KeaRNS-Sayre Syndrome (KSS); Lactic Acidosis; Long-Chain Acyl-CoA Dehydrogenase Deficiency (LCAD); LCHAD; Leigh Syndrome; Leigh-like Syndrome; Leber's Hereditary Optic Neuropathy (LHON); Lethal Infantile Cardiomyopathy (LIC); Luft Disease; Multiple Acyl-CoA Dehydrogenase Deficiency (MAD); Medium-Chain Acyl-CoA Dehydrogenase Deficiency (MCAD); Mitochondrial Myopathy, Encephalopathy, Lactacidosis, Stroke (MELAS); Myoclonic Epilepsy with Ragged Red Fibers (MERRF); Mitochondrial Recessive Ataxia Syndrome (MIRAS); Mitochondrial Cytopathy, Mitochondrial DNA Depletion; Mitochondrial Encephalopathy; Mitochondrial Myopathy; Myoneurogastointestinal Disorder and Encephalopathy (MNGIE); Neuropathy, Ataxia, and Retinitis Pigmentosa (NARP); Pearson Syndrome; Pyruvate Carboxylase Deficiency; Pyruvate Dehydrogenase Deficiency; a Respiratory Chain Disorder; Short-Chain Acyl-CoA Dehydrogenase Deficiency (SCAD); SCHAD; Very Long-Chain Acyl-CoA Dehydrogenase Deficiency (VLCAD); a myopathy; cardiomyopathy; encephalomyopathy; a neurodegenerative disease; Parkinson's disease; Alzheimer's disease; amyotrophic lateral sclerosis (ALS); a motor neuron disease; a neurological disease; epilepsy; an age-associated disease; macular degeneration; diabetes; metabolic syndrome; brain cancer; a genetic disease; Huntington's Disease; a mood disorder; schizophrenia; bipolar disorder; a pervasive developmental disorder; autistic disorder; Asperger's syndrome; childhood disintegrative disorder (CDD); Rett's disorder; PDD-not otherwise specified (PDD-NOS); a cerebrovascular accident; stroke; a vision impairment; optic neuropathy; dominant inherited juvenile optic atrophy; optic neuropathy caused by a toxic agent; glaucoma; Stargardt's macular dystrophy; diabetic retinopathy; diabetic maculopathy; retinopathy of prematurity; ischemic reperfusion related retinal injury; oxygen poisoning; a haemoglobionopathy; thalassemia; sickle cell anemia; seizures; ischemia; renal tubular acidosis; attention deficit/hyperactivity disorder (ADHD); a neurodegenerative disorder resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; a muscular dystrophy; a leukodystrophy; Progressive Supranuclear Palsy; spinal muscular atrophy; hearing loss; noise induced hearing loss; traumatic brain injury; Juvenile Huntington's Disease; Multiple Sclerosis; NGLY1; Multisystem atrophy; Adrenoleukodystrophy; Adrenomyeloneuropathy; and any combination thereof.

In some embodiments, the redox-modulating composition improves one or more energy biomarkers in the subject or the redox-sensitive composition selected from the group consisting of: lactic acid (lactate) levels; pyruvic acid (pyruvate) levels; lactate/pyruvate ratios; total, reduced or oxidized glutathione levels, or reduced/oxidized glutathione ratio; total, reduced or oxidized cysteine levels, or reduced/oxidized cysteine ratio; phosphocreatine levels, NADH (NADH+H⁺) levels; NADPH (NADPH+H⁺) levels; NAD levels; NADP levels; ATP levels; reduced coenzyme Q (CoQred) levels; oxidized coenzyme Q (CoQox) levels; total coenzyme Q (CoQtot) levels; oxidized cytochrome C levels; reduced cytochrome C levels; oxidized cytochrome C/reduced cytochrome C ratio; acetoacetate levels, b-hydroxy butyrate levels, acetoacetate/b-hydroxy butyrate ratio, 8-hydroxy-2′-deoxyguanosine (8-OHdG) levels; levels of reactive oxygen species; levels of oxygen consumption (V02); levels of carbon dioxide output (VC02); respiratory quotient (VC02/V02); exercise tolerance; anaerobic threshold; and any combination thereof.

### BRIEF DESCRIPTION OF THE DRAWINGS

FIGS. 1A-1B depict data related to the effect of second organelle complexes (2^{nd} OC) on ejection fraction (EF) in swine with ischemia-reperfusion injury. FIG. 1A depicts changes in EF (%) (Control, N=5-3, two dead before day 1; 250 μg second organelle complexes, N=4). FIG. 1B depicts the percentage change from pre-ischemia treatment 15 minutes after reperfusion (control, N=5, 250 μg second organelle complexes, N=4). Data are expressed mean±SEM.

FIGS. 2A-2B depict the experimental setup (FIG. 2A) and results from a luminol assay (FIG. 2B) comparing anti-ROS activity between second organelle complexes (2^{nd} OC) and homogenized mitochondria (H-mito).

FIGS. 3A-3B depict the experimental setup (FIG. 3A) and data (FIG. 3B) related to NOC7-DAF-2 assays with 100 μg/mL of the indicated populations.

FIGS. 4A-4B depict the experimental setup (FIG. 4A) and data (FIG. 4B) related to cell-based assays examining the in vivo redox-modulating activity of the compositions provided herein. FIG. 4B shows the results of the assay (performed in triplicate) with second organelle complexes (2^{nd} OC) derived from HeLa cells.

FIGS. 5A-5B depict the experimental setup (FIG. 5A) and data (FIG. 5B) related to CellTiter-Glo® 2.0 assays examining the in vitro redox-modulating activity of the compositions provided herein with HUEhT2 cells. Similar results were observed with Raw264.7 cells (not shown).

FIGS. 6A-6B depict the experimental setup (FIG. 6A) and data (FIG. 6B) related to cell viability assays examining the redox-modulating compositions provided herein. The first organelle complexes (1^{st} OC) derived from HeLa cells prevents cell death induced by H₂O₂ treatment for 2 hours. The cell viability results are expressed by ratio to control (without H₂O₂ treatment).

FIGS. 7A-7B depict the experimental setup (FIG. 7A) and data (FIG. 7B) related to NAD⁺/NADH assays examining the redox-modulating compositions provided herein. The first organelle complexes (1^{st} OC) derived from HeLa cells on NAD⁺/NADH ratios 2 hours after contact with H₂O₂.

FIGS. 8A-8B depict the experimental setup (FIG. 8A) and data (FIG. 8B) related to CellTiter-Glo® 2.0 assays comparing first organelle complexes (1^{st} OC) and second organelle complexes (2^{nd} OC) derived from HeLa cells. The first and second organelle complexes prevents cell death 2 hours (not shown), 4 hours (not shown) and 24 hours after contact with H₂O₂. The cell viability results are expressed by ratio to PBS control (without H₂O₂ treatment).

FIG. 9 depicts data related to characterization of the organelle complexes populations provided herein. Intracellular structures/organelles western blot protein analysis of first organelle complexes (1^{st} OC) and second organelle complexes (2^{nd} OC) prepared using the methods disclosed herein from HEK293T cells.

FIGS. 10A-10C depict data related to the role of GSH in the anti-ROS activity of first organelle complexes. FIG. 10A depicts GSH concentration in first organelle complexes derived from HEK293 cells that have (BSO-1^{st} OC) or have not (293-1^{st} OC) been contacted with buthionine sulfoximine (BSO). FIGS. 10B-10C depict CellTiter-Glo cell viability results after HEK293 cells were incubated with H₂O₂ (100 μM) and either 293-1^{st} OC or BSO-1^{st} OC for 4 hours (FIG. 10B) or 20 hours (FIG. 10C).

FIGS. 11A-11C depict data related to the role of catalase in the anti-ROS activity of first organelle complexes. FIG. 11A depicts data related to the knockdown of catalase (CAT) in HEK 293 cells after transfection with catalase siRNA for 24 hours or 48 hours (catalase knockdown is highlighted by box; vinculin is the loading control). FIG. 11B depicts catalase levels in untreated first organelle complexes (1^{st} OC) or catalase-depleted first organelle complexes (1^{st} OC-siRNA; knockdown highlighted by box). FIG. 11C depicts CellTiter-Glo cell viability results of HEK293 cells (n=3) preincubated with either first organelle complexes (293-1^{st} OC) or catalase-depleted first organelle complexes (293-1^{st} OC-siRNA) for 6 hours, followed by 18 hours of H₂O₂ (300 μM) treatment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2^{nd} ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2^{nd} ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “isolated” shall be given its ordinary meaning and shall also refer to a substance or entity that has been (1) separated from at least some of the components with which it was associated when initially produced (whether in nature or in an experimental setting), and/or (2) produced, prepared, and/or manufactured by the hand of man. In some embodiments, an isolated mitochondrion or isolated organelle complexes population has been processed to obtain it from a cellular environment via the methods provided herein.

As used herein, the term “cell” shall be given its ordinary meaning and shall also refer to a eukaryotic cell, i.e., a cell that contains mitochondria in the cytoplasm, e.g., an animal cell, e.g., a mammalian cell, preferably a human cell. As used herein, the term “cell” is used in the meaning to include a cell present in a tissue, and a cell separated from a tissue (e.g., a single cell), and a cell that is within a population of cells (e.g., a population of cells obtained from a tissue of a subject, and/or a population of cells obtained from a cell line.

As used herein, the term “mitochondrion” shall be given its ordinary meaning and shall also refer to an organelle present in a eukaryotic cell that has double-layered lipid membranes, the inner and outer membranes, and a matrix surrounded by cristae and inner membranes. Mitochondria (more than one mitochondrion) have enzymes on their inner membrane, such as the respiratory chain complexes, which is involved in oxidative phosphorylation. The inner membrane has a membrane potential due to the internal-external proton gradients formed by the action of the respiratory chain complexes, etc. Mitochondria are thought to be unable to maintain the membrane potential when the inner membrane is disrupted. Mitochondria are known to have their own genomes (mitochondrial genomes) that differ from the genome in the cell nucleus.

As used herein, the term “organelle complex” shall be given its ordinary meaning and shall also refer to a complex of mitochondria and one or more of endoplasmic reticulum, peroxisomes, lysosomes, and Golgi apparatus. Organelle complexes can be depleted of cytosolic macromolecules (e.g., cytosolic proteins). In some embodiments, organelle complexes do not comprise cytosolic macromolecules. In some embodiments, an organelle complexes population comprises homogenized mitochondria. As used herein, the term “population” shall be given its ordinary meaning and shall also refer to a group of a plurality of the same or different substances. For example, an “organelle complexes population” is a group of at least a plurality of the same or different organelle complexes. The population may not be always homogenous and may have physical, chemical and/or physiological distributions. The physical distribution includes, for example, particle size and polydispersity index. The chemical distribution includes, for example, a zeta potential distribution and a lipid composition distribution. The physiological distribution includes, for example, a difference of physiological function (for example, respiratory activity). An organelle complexes population can comprise first organelle complexes, second organelle complexes, homogenized mitochondria, or any combination thereof. As used herein, the term “homogenized mitochondria” shall be given its ordinary meaning and shall also refer to mitochondria isolated via a method comprising one or more homogenization steps.

As used herein, the term “surfactant” shall be given its ordinary meaning and shall also refer to a molecule having a hydrophilic moiety and a hydrophobic moiety in one molecule. Surfactants have the role of reducing surface tension at the interface or mixing polar and non-polar substances by forming micelles. Surfactants are roughly classified into nonionic surfactants and ionic surfactants. Nonionic surfactants are those in which the hydrophilic moiety is not ionized, and ionic surfactants are those in which the hydrophilic moiety comprises either a cation or an anion or both a cation and an anion.

As used herein, the term “critical micelle concentration” (CMC) shall be given its ordinary meaning and shall also refer to the concentration at which, when the concentration is reached, the surfactant forms micelles, and the surfactant further added to the system contributes to micelle formation, in particular the concentration in bulk. At concentrations above the critical micelle concentration, the addition of surfactants to the system ideally increases the amount of micelles, especially the number of micelles.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “oxidative stress” shall be given its ordinary meaning and shall also refer to an imbalance between generation of reactive oxygen species, reactive nitrogen species, and/or free radicals, and the antioxidative capacity of biological system.

As used herein, the term “reductive stress” shall be given its ordinary meaning and shall also refer to a response to conditions that shift the redox balance of important biological redox couples, such as the NAD⁺/NADH, NADP⁺/NADPH, and GSH/GSSG, to a more reducing state. In some embodiments, reductive stress is the counterpart oxidative stress.

As used herein, the term “effective amount” refers to an amount sufficient to effect beneficial or desirable biological and/or clinical results.

The methods, compositions, systems, and kits provided herein can, in some embodiments, be employed in concert with the methods, compositions, systems, and kits described in PCT Patent Application Publication Nos. WO2018/092839, WO2017/090763, WO2020/230601, WO2019/164003, WO2020/054824, WO2020/203961, WO2020/054829, WO2021/015298, and WO2021/132735, the contents of which are incorporated herein by reference in their entirety.

### Redox-Modulating Mitochondria

There are provided, in some embodiments, methods of reducing or preventing oxidative stress and/or reductive stress in a redox-sensitive composition. In some embodiments, the method comprises: contacting a redox-sensitive composition with an effective amount of a redox-modulating composition, thereby reducing or preventing oxidative stress and/or reductive stress in the redox-sensitive composition.

There are provided, in some embodiments, methods of reducing levels of reactive oxygen species (ROS) and/or reactive nitrogen species (RNS) in a redox-sensitive composition. In some embodiments, the method comprises: contacting a redox-sensitive composition with an effective amount of a redox-modulating composition, thereby reducing levels of ROS and/or RNS in the redox-sensitive composition. In some embodiments, reducing levels of ROS and/or RNS in the redox-sensitive composition thereby reduces or prevents oxidative stress and/or reductive stress in the redox-sensitive composition.

The contacting step can comprise incubating a mixture of the redox-sensitive composition and the redox-modulating composition for less than about 30 seconds (e.g., 30 secs, 25 secs, 20 secs, 15 sec, 10 secs, 5 secs, 1 sec, 1 millisecond, or a number or a range between any of these values). The redox-sensitive composition can be experiencing oxidative stress and/or reductive stress or can be at risk of experiencing oxidative stress and/or reductive stress. The redox-sensitive composition can comprise one or more cells. The contacting step can comprise introducing the redox-modulating composition into the one or more cells. The one or more cells can be cells of a subject. The one or more cells can be undergoing or at risk of undergoing hypoxia. The contacting can be performed ex vivo, in vitro, or in vivo.

The redox-sensitive composition can comprise a biological sample. The biological sample can be selected from the group consisting of a soil sample, an air sample, an environmental sample, a cell culture sample, a bone marrow sample, a rainfall sample, a fallout sample, a space sample, an extraterrestrial sample, a sewage sample, a ground water sample, an abrasion sample, an archaeological sample, a food sample, a blood sample, a serum sample, a plasma sample, a urine sample, a stool sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumor sample, a tissue sample, a cell sample, a cell lysate sample, a virus culture sample, a forensic sample, an infection sample, a nosocomial infection sample, a production sample, a drug preparation sample, a biological molecule production sample, a protein preparation sample, a lipid preparation sample, a carbohydrate preparation sample, a solution of a nucleotide, a solution of polynucleotide, a solution of a nucleic acid, a solution of a peptide, a solution of a polypeptide, a solution of an amino acid, a solution of a protein, a solution of a synthetic polymer, a solution of a biochemical composition, a solution of an organic chemical composition, a solution of an inorganic chemical composition, a solution of a lipid, a solution of a carbohydrate, a solution of a combinatory chemistry product, a solution of a drug candidate molecule, a solution of a drug molecule, a solution of a drug metabolite, a suspension of a cell, a suspension of a virus, a suspension of a microorganism, a suspension of a metal, a suspension of metal alloy, a solution of a metal ion, and any combination thereof.

There are provided, in some embodiments, methods of reducing or preventing oxidative stress and/or reductive stress in a subject. In some embodiments, the method comprises: administering to the subject an effective amount of a redox-modulating composition, thereby reducing or preventing oxidative stress and/or reductive stress in the subject.

There are provided, in some embodiments, methods of treating or preventing a redox disease or disorder in a subject. In some embodiments, the method comprises: administering to the subject an effective amount of a redox-modulating composition, thereby treating or preventing a redox disease or disorder in a subject.

The redox-modulating composition can comprise an organelle complexes population. The organelle complexes population can comprise first organelle complexes, or a combination of first organelle complexes and second organelle complexes. The redox-modulating composition can comprise isolated organelle complexes. The redox-modulating composition can comprise homogenized mitochondria, first organelle complexes, and/or second organelle complexes. The organelle complexes can comprise mitochondria and one or more of endoplasmic reticulum, peroxisomes, lysosomes, and Golgi apparatus. The first organelle complexes and/or second organelle complexes can be depleted of cytosolic macromolecules. The redox-modulating composition can comprise isolated mitochondria. The redox-modulating composition can comprise mitochondria isolated from intact cells and/or floating cells or frozen cells or combination thereof. In some embodiments, the redox-modulating composition does not comprise intact cells. The organelle complexes (e.g., first organelle complexes, second organelle complexes) provided herein can comprise mitochondria and one, two, three, or four of endoplasmic reticulum, peroxisomes, lysosomes, and Golgi apparatus. The organelle complexes (e.g., first organelle complexes, second organelle complexes) can comprise: (i) mitochondria and endoplasmic reticulum; (ii) mitochondria and peroxisomes; (iii) mitochondria and lysosomes; (iv) mitochondria and Golgi apparatus; (v) mitochondria, endoplasmic reticulum, and peroxisomes; (vi) mitochondria, endoplasmic reticulum, and lysosomes; (vii) mitochondria, endoplasmic reticulum, and Golgi apparatus; (viii) mitochondria, endoplasmic reticulum, peroxisomes, and lysosomes; (ix) mitochondria, endoplasmic reticulum, peroxisomes, and Golgi apparatus; (x) mitochondria, endoplasmic reticulum, peroxisomes, lysosomes, and Golgi apparatus; (xi) mitochondria, endoplasmic reticulum, lysosomes, and Golgi apparatus; (xii) mitochondria, peroxisomes, and lysosomes; (xiii) mitochondria, peroxisomes, and Golgi apparatus; (xiv) mitochondria, peroxisomes, lysosomes, and Golgi apparatus; and/or (xv) mitochondria, lysosomes, and Golgi apparatus. The ratio of mitochondria to additional organelles (e.g., endoplasmic reticulum, peroxisomes, lysosomes, and/or Golgi apparatus) in the organelle complexes population can vary.

Disclosed herein include methods for generating first organelle complexes populations. In some embodiments, the method comprises: incubating cells in a first solution comprising a surfactant at a first temperature; removing the surfactant to form a second solution; and recovering first organelle complexes from the second solution. First organelle complexes can be derived from: (i) frozen cells; (ii) floating cells; and/or (iii) cells contacted with a surfactant at a concentration at or above the critical micellar concentration (CMC) for the surfactant. There are provided, in some embodiments, second organelle complexes. In some embodiments, the method for isolating second organelle complexes from cells comprises treating cells in a first solution with a surfactant at a concentration below the critical micelle concentration (CMC) for the surfactant, removing the surfactant to form a second solution, incubating the cells in the second solution, and recovering second organelle complexes from the second solution. Second organelle complexes can be derived from: (i) adherent cells; and/or (ii) cells contacted with a surfactant at a concentration below the critical micellar concentration (CMC) for the surfactant. The organelle complexes population can be derived from cells treated with a mitochondria-activating agent (e.g., resveratrol). The organelle complexes can be depleted of cytosolic macromolecules. Cytosolic macromolecules can be absent from the organelle complexes populations provided herein. Organelle complexes (e.g., first organelle complexes, second organelle complexes) populations provided herein can comprise a negligible and/or undetectable amount of cytosolic macromolecules. The redox-modulating composition can comprise a substantially pure organelle complexes population. A substantially pure organelle complexes population can comprise less than about 20% (e.g., less than about 20%, 18%, 16%, 14%, 12%, 10%, 8%, 6%, 4%, 2%, 1%, 0.1%, 0.01%, 0.001%, 0%, or a number or a range between any two of the values) cytosolic macromolecules. The cytosolic macromolecules can comprise cytosolic proteins (e.g., p70S6K and/or glyceraldehyde 3-phosphate dehydrogenase (GAPDH)). The first organelle complexes and second organelle complexes can be derived from cells treated with a mitochondria-activating agent. The homogenized mitochondria, first organelle complexes, and/or second organelle complexes can be encapsulated in lipid membrane-based vesicles. Methods of encapsulating in lipid membrane-based vesicles are disclosed in PCT Patent Application Publication No. WO2021/132735, the contents of which are incorporated herein by reference in its entirety. Incubating cells in the first solution and/or incubating the second solution can comprise applying a physical stimulus to the first solution and/or the second solution, respectively, such as, for example, pipetting, shaking and/or stirring. Applying a physical stimulus to the first solution and/or the second solution can comprise flowing the first solution and/or the second solution through a flow device (e.g., a reducer flow device). Said flow device can comprise a fluidic channel comprising two or more segments of varying cross-sectional diameters. Recovering the first organelle complexes from the second solution can comprise tangential flow filtration (TFF). The systems, methods, compositions, and kits provided herein can, in some embodiments, be employed in concert with the systems, methods, compositions, and kits for generating first organelle complexes described in PCT Patent Application No. PCT/US23/27014, entitled, “ORGANELLE COMPLEXES,” filed Jul. 6, 2023, the content of which is incorporated herein by reference in its entirety.

The method can comprise at least about 5 ug/mL to about 5 mg/mL of the redox-sensitive composition. The effective amount can comprise at least about 1 ug to about 1 mg of the redox-modulating composition. The effective amount can comprise at least about 5 ug/mL to about 5 mg/mL of the redox-modulating composition. The amount of the redox-sensitive composition and/or the effective amount of the redox-modulating composition can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, ug, mg, ug/mL, and/or mg/mL.

The oxidative stress and/or reductive stress can comprise elevated levels of ROS, RNS, and/or free radicals. The oxidative stress and/or reductive stress can comprise altered cell functions. The oxidative stress and/or reductive stress can be associated with a redox disease or disorder. In some embodiments, the redox-modulating composition reduces levels of one or more ROS and/or one or more RNS in the subject or the redox-sensitive composition by at least about 5% (e.g., 5%, 6%, 7%, 8%, 9%, 10%, 11%, 12%, 13%, 14%, 15%, 16%, 17%, 18%, 19%, 20%, 25%, 30%, 40%, 50%, 75%, 100%, or higher and overlapping ranges therein). In some embodiments, the redox-modulating composition reduces level of oxidative cell stress and thereby recovers or restores the cell functions.

The redox-modulating composition can have a ROS scavenging activity and/or RNS scavenging activity. In some embodiments, the redox-modulating composition reduces or prevents ROS generation and/or RNS generation in the subject. The redox-modulating composition can have superoxide dismutase activity, catalase activity, peroxidase activity, or any combination thereof. The redox-modulating composition can have free radical scavenging activity and/or inhibits free radical formation.

In some embodiments, the redox-modulating composition increases and/or decreases (by least about 1.1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) one or more of the following ratios in the subject or the redox-sensitive composition: oxidized to reduced forms of nicotinamide adenine dinucleotide (NAD⁺/NADH), oxidized to reduced forms of nicotinamide adenine dinucleotide phosphate (NADP⁺/NADPH), oxidized to reduced forms of glutathione (GSSG/GSH), and oxidized to reduced forms of thioredoxin (TrxSS/TrxSH₂).

The reactive nitrogen species can comprise nitric oxide (NO), nitrogen dioxide radical (·NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) or dinitrogen trioxide (N₂O₃), or any combination thereof. The reactive oxygen species can comprise superoxide (O₂·⁻), hydroperoxy (HO·₂), hydrogen peroxide (H₂O₂), peroxynitrite (ONOO⁻), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof. The ROS can be derived from neutrophils and/or xanthine oxidase (XO).

The redox-modulating composition can, via an antioxidant effect and/or a reductive effect, protect against ROS-elicited functional changes. The redox-modulating composition can be an antioxidant composition, an anti-reductant composition, or a combination thereof. The redox-sensitive composition can be an oxidant-sensitive composition, a reductant-sensitive composition, or a combination thereof. The protection mediated by the redox-modulating compositions provided herein can be intracellular, extracellular, and/or in a cell-free environment. The redox-modulating composition can improve (by least about 1.1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) one or more energy biomarkers in the subject or the redox-sensitive composition selected from the group consisting of: lactic acid (lactate) levels; pyruvic acid (pyruvate) levels; lactate/pyruvate ratios; total, reduced or oxidized glutathione levels, or reduced/oxidized glutathione ratio; total, reduced or oxidized cysteine levels, or reduced/oxidized cysteine ratio; phosphocreatine levels, NADH (NADH+H⁺) levels; NADPH (NADPH+H⁺) levels; NAD levels; NADP levels; ATP levels; reduced coenzyme Q (CoQred) levels; oxidized coenzyme Q (CoQox) levels; total coenzyme Q (CoQtot) levels; oxidized cytochrome C levels; reduced cytochrome C levels; oxidized cytochrome C/reduced cytochrome C ratio; acetoacetate levels, b-hydroxy butyrate levels, acetoacetate/b-hydroxy butyrate ratio, 8-hydroxy-2′-deoxyguanosine (8-OHdG) levels; levels of reactive oxygen species; levels of oxygen consumption (V02); levels of carbon dioxide output (VC02); respiratory quotient (VC02/V02); exercise tolerance; anaerobic threshold; and any combination thereof. The redox-modulating compositions provided herein can increase lactate and/or ATP production in a dose-dependent manner (by least about 1.1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, and without being bound by any particular theory, the redox-modulating composition modulates the ratio of nicotinamide adenine dinucleotide, reduced form (NADH) to nicotinamide adenine dinucleotide, oxidized form (NAD⁺). In some embodiments, and without being bound by any particular theory, the redox-modulating composition modulates the ratio of nicotinamide adenine dinucleotide phosphate, reduced form (NADPH) to nicotinamide adenine dinucleotide phosphate, oxidized form (NADP⁺). NAD⁺/NADH can participate in redox reactions in energy metabolism and mitochondria function. NADH/NADPH can participate in ROS metabolism as an electron donor. Various methods for measuring intracellular NAD⁺ are known in the art, and include LC-MS/MS, HPLC, NMR, MS imaging, in situ genetically-encoded sensors, MRI assays, lysate-based approaches, and others, such as those provided in Cambronne X A, Kraus W L. Trends Biochem Sci. 2020 October; 45(10)/858-873, the content of which is hereby incorporated by reference in its entirety. The redox-modulating composition can exert a reductive effect on the redox-sensitive composition and/or cells of a subject. The redox-modulating composition can reduce the generation of RNS and/or ROS in the redox-sensitive composition or subject. The redox-modulating composition can increase the removal of RNS and/or ROS in a redox-sensitive composition or a subject. In some embodiments, redox-modulating compositions comprising first organelle complexes can reduce the generation of and/or increase the removal of RNS and/or ROS in a redox-sensitive composition or a subject by at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more than redox-modulating compositions comprising second organelle complexes. The redox-modulating compositions provided herein can influence the redox system and thereby cause increased viability (e.g., via an antioxidant effect and/or anti-reductant effect). In some embodiments, excessive or lethal oxidative stress is a reductive stress, and in some such embodiments, it requires not only a reducing action by antioxidant action, but also an oxidizing action by anti-reducing action. The disclosed redox-modulating compositions can be an antioxidant stress action and an anti-reductive stress action, e.g., a REDOX enhancer. Reductive stress can comprise a high Lactate/Pyruvate ratio. The redox-modulating compositions provided herein can act via decreasing the level of excess reducing equivalents. In some embodiments, and without being bound by any particular theory, the redox-modulating compositions disclosed herein provide scavenging of reductive stress and can reduce reductive stress by oxidizing NADH to NAD+, which can lead to antioxidant activity, ATP production capacity, and/or cell proliferative capacity. In some embodiments, and without being bound by any particular theory, the redox-modulating compositions provided herein can modulate (e.g., increase and/or decrease) the ratio of one or more of NAD⁺/NADH, NADP⁺/NADPH, and GSH/GSSG by at least 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), thereby alleviating reductive stress. In some embodiments, and without being bound by any particular theory, the oxidized NAD⁺ regulates NADPH-linked redox systems and/or acts as a signaling molecule for homeostasis. The redox-modulating compositions provided herein (e.g., organelle complexes) can be associated with redox systems (including those involved in ROS generation and/or ROS clearance) such as, for example, peroxisomes, mitochondria, endosomes (e.g., plasma membrane endosomes), and endoplasmic reticulum. In some embodiments, organelle complexes contain more NAD⁺. In some embodiments, NAD⁺ is an active ingredient of the redox-modulating compositions provided herein. In some embodiments, first organelle complexes contain at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more NAD⁺ than second organelle complexes. In some embodiments, removing NAD⁺ from the redox-modulating compositions provided herein reduce at least a portion of the effect exerted by the redox-modulating composition.

In embodiments, the organelle complexes population may treat, prevent, ameliorate, and/or improve clinical condition due to ischemia-reperfusion injury. In embodiments, the organelle complexes population may improve Ejection Fraction (EF), inhibit cardiac hypertrophy, and/or treat, prevent, ameliorate, and/or improve fibrosis after ischemia-reperfusion injury. The redox-modulating composition can improve (by least about 1.1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) one or more heart function indicators in the subject selected from the group consisting of cardiac output, ejection fraction, volumes, stroke volume, pressures, end-diastolic volume (EDV), and end-systolic volume (ESV).

### Pharmaceutically Acceptable Compositions and Methods of Administration

In some embodiments, the redox-modulating composition is administered to the subject in combination with one or more additional agents and/or additional therapies designed to treat the disease or disorder. In some embodiments, the present disclosure provides methods for treating diseases and disorders associated with mitochondrial dysfunction or diseases or disorders that otherwise benefit from the supplementation of healthy, functional mitochondria. The present disclosure also provides use of redox-modulating composition in the manufacture of a medicament for treating the diseases and disorders provided herein.

The administering can comprise intravenous administration, intra-arterial administration, intra-tracheal administration, subcutaneous, administration intramuscular administration, inhalation, intrapulmonary administration, and/or intraocular administration. The redox-modulating composition can be administered locally or systemically.

The wording “local administration” or “topic administration” as used herein indicates any route of administration by which a redox-modulating composition is brought in contact with the body of the individual, so that the resulting redox-modulating composition location in the body is topic (limited to a specific tissue, organ or other body part where the imaging is desired). Exemplary local administration routes include injection into a particular tissue by a needle, gavage into the gastrointestinal tract, and spreading a solution containing redox-modulating composition on a skin surface.

The wording “systemic administration” as used herein indicates any route of administration by which a redox-modulating composition is brought in contact with the body of the individual, so that the resulting redox-modulating composition location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In another aspect, this disclosure provides pharmaceutically acceptable compositions which comprise a therapeutically-effective amount of a redox-modulating composition disclosed herein. As described in detail below, the pharmaceutical compositions of this disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the redox-modulating composition. The pharmaceutical compositions can comprise one or more pharmaceutically-acceptable carriers. The phrase “therapeutically-effective amount” as used herein can refer to that amount of a redox-modulating composition disclosed herein which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of this disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., redox-modulating composition) which can be combined with a carrier material to produce a single dosage form will vary depending upon the host being treated, the particular mode of administration. The amount of active ingredient, which can be combined with a carrier material to produce a single dosage form will generally be that amount of the redox-modulating composition which produces a therapeutic effect. Generally, out of one hundred percent, this amount will range from about 1% to about 99% of active ingredient, preferably from about 5% to about 70%, most preferably from about 10% to about 30%.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a redox-modulating composition include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions and the like, are also contemplated as being within the scope of this disclosure.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Actual dosage levels of the active ingredients in the pharmaceutical compositions of this disclosure may be determined by the methods of this disclosure so as to obtain an amount of the active ingredient, which is effective to achieve the desired therapeutic response for a particular subject, composition, and mode of administration, without being toxic to the subject.

The subject can be experiencing oxidative stress and/or reductive stress or can be at risk of experiencing oxidative stress and/or reductive stress (e.g., oxidative stress and/or reductive stress caused by ischemic-reperfusion injury or the redox disease or disorder). In some embodiments, the method comprises: identifying a subject experiencing oxidative stress and/or reductive stress or at risk of experiencing oxidative stress and/or reductive stress. In some embodiments, the method comprises: measuring the levels of RNS in the subject or the redox-sensitive composition with a probe that detects reactive nitrogen species; and/or measuring the levels of ROS in the subject or the redox-sensitive composition with a probe that detects reactive oxygen species. The probe that detects reactive nitrogen species can be diaminonaphthalene, a diaminofluorescein, a diaminorhodamine, a diaminocyanine, an NiSPY, dichlorodiaminocalcein, 1,2-diaminoanthraquinone, or DAMBO-P^{H}. The probe that detects reactive oxygen species can be 2′,7′-dichloro-fluorescein diacetate, dihydrorhodamine 123, 3′-(p-aminophenyl) fluorescein (API), 3′-(p-hydroxyphenyl) fluorescein (HPF), aminophenoxycalcein (APC), mitoAR, mitoHR, DPAX, DMAX, a hydrocyanine, or dihydroethidium.

The subject can have or can be suspected of having a disease or condition in which ROS is implicated selected from the group consisting of atherosclerosis, heart disease, heart failure, hypertension, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, stroke, an ischemia-reperfusion injury, an ischemia-reperfusion injury, chronic inflammation, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack, UV damage to the skin, organ transplantation, surgery, and any combination thereof.

In some embodiments, administering the redox-modulating composition to the subject treats, reduces, or prevents ischemia-reperfusion injury of the subject. The ischemic-reperfusion injury can be caused by mitochondrial dysfunction, hypoxic injury, HMGB1 release or necrotic cell death. In some embodiments, the redox-modulating composition suppresses ischemic-reperfusion injury, mitochondrial dysfunction, hypoxic injury, necrotic cell death, or any combination thereof.

The redox disease or disorder can comprise ROS-mediated and/or RNS-mediated oxidative damage to one or more tissues of the subject. The redox disease or disorder can be selected from the group consisting of: a mitochondrial disorder; an inherited mitochondrial disease; Alpers Disease; Barth syndrome; a Beta-oxidation Defect; Carnitine-Acyl-Carnitine Deficiency; Carnitine Deficiency; a Creatine Deficiency Syndrome; Co-Enzyme Q10 Deficiency; Complex I Deficiency; Complex II Deficiency; Complex III Deficiency; Complex IV Deficiency; Complex V Deficiency; COX Deficiency; chronic progressive external ophthalmoplegia (CPEO); CPT I Deficiency; CPT II deficiency; Friedreich's Ataxia (FA); Glutaric Aciduria Type II; KeaRNS-Sayre Syndrome (KSS); Lactic Acidosis; Long-Chain Acyl-CoA Dehydrogenase Deficiency (LCAD); LCHAD; Leigh Syndrome; Leigh-like Syndrome; Leber's Hereditary Optic Neuropathy (LHON); Lethal Infantile Cardiomyopathy (LIC); Luft Disease; Multiple Acyl-CoA Dehydrogenase Deficiency (MAD); Medium-Chain Acyl-CoA Dehydrogenase Deficiency (MCAD); Mitochondrial Myopathy, Encephalopathy, Lactacidosis, Stroke (MELAS); Myoclonic Epilepsy with Ragged Red Fibers (MERRF); Mitochondrial Recessive Ataxia Syndrome (MIRAS); Mitochondrial Cytopathy, Mitochondrial DNA Depletion; Mitochondrial Encephalopathy; Mitochondrial Myopathy; Myoneurogastointestinal Disorder and Encephalopathy (MNGIE); Neuropathy, Ataxia, and Retinitis Pigmentosa (NARP); Pearson Syndrome; Pyruvate Carboxylase Deficiency; Pyruvate Dehydrogenase Deficiency; a Respiratory Chain Disorder; Short-Chain Acyl-CoA Dehydrogenase Deficiency (SCAD); SCHAD; Very Long-Chain Acyl-CoA Dehydrogenase Deficiency (VLCAD); a myopathy; cardiomyopathy; encephalomyopathy; a neurodegenerative disease; Parkinson's disease; Alzheimer's disease; amyotrophic lateral sclerosis (ALS); a motor neuron disease; a neurological disease; epilepsy; an age-associated disease; macular degeneration; diabetes; metabolic syndrome; brain cancer; a genetic disease; Huntington's Disease; a mood disorder; schizophrenia; bipolar disorder; a pervasive developmental disorder; autistic disorder; Asperger's syndrome; childhood disintegrative disorder (CDD); Rett's disorder; PDD-not otherwise specified (PDD-NOS); a cerebrovascular accident; stroke; a vision impairment; optic neuropathy; dominant inherited juvenile optic atrophy; optic neuropathy caused by a toxic agent; glaucoma; Stargardt's macular dystrophy; diabetic retinopathy; diabetic maculopathy; retinopathy of prematurity; ischemic reperfusion related retinal injury; oxygen poisoning; a haemoglobionopathy; thalassemia; sickle cell anemia; seizures; ischemia; renal tubular acidosis; attention deficit/hyperactivity disorder (ADHD); a neurodegenerative disorder resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; a muscular dystrophy; a leukodystrophy; Progressive Supranuclear Palsy; spinal muscular atrophy; hearing loss; noise induced hearing loss; traumatic brain injury; Juvenile Huntington's Disease; Multiple Sclerosis; NGLY1; Multisystem atrophy; Adrenoleukodystrophy; Adrenomyeloneuropathy; and any combination thereof.

Also provided herein are kits comprising one or more compositions (e.g., a formulation comprising a redox-modulating composition) described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

### EXAMPLES

Some aspects of the embodiments discussed above are disclosed in further detail in the following examples, which are not in any way intended to limit the scope of the present disclosure.

### Example 1

### Effect of Second Organelle Complexes on Ejection Fraction in Swine With Ischemia-Reperfusion Injury

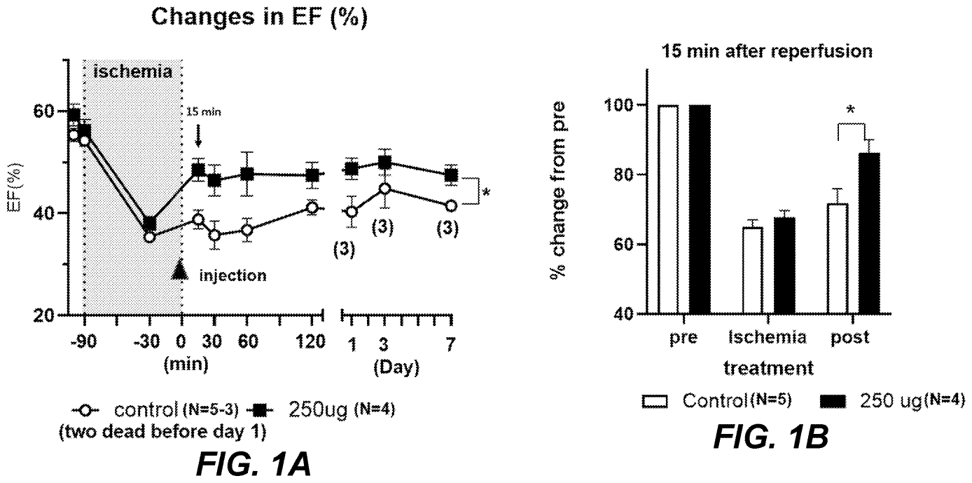

The effect of second organelle complexes on ejection fraction in swine with ischemia-reperfusion injury was investigated. Ischemia was elicited by inflation of balloon placed at level of D1 in LAD. Second organelle complexes were given into LAD peripheral to inflation site immediately before reperfusion. FIGS. 1A-1B depict data related to the effect of second organelle complexes (2^{nd} OC) on ejection fraction (EF) in swine with ischemia-reperfusion injury. FIG. 1A depicts changes in EF (%) (Control, N=5-3, two dead before day 1; 250 μg second organelle complexes, N=4). FIG. 1B depicts the percentage change from pre-ischemia treatment 15 minutes after reperfusion (control, N=5, 250 μg second organelle complexes, N=4). Data are expressed mean±SEM. ROS and RNS production has been implicated in ischemia-reperfusion injury (See Int. J. Mol. Sci. 2018, 19, 417; doi:10.3390/ijms19020417, incorporated herein by reference). These data indicate a redox-modulating effect of second organelle complexes disclosed herein.

### Example 2

### Anti-ROS Activity of Redox-Modulating Compositions

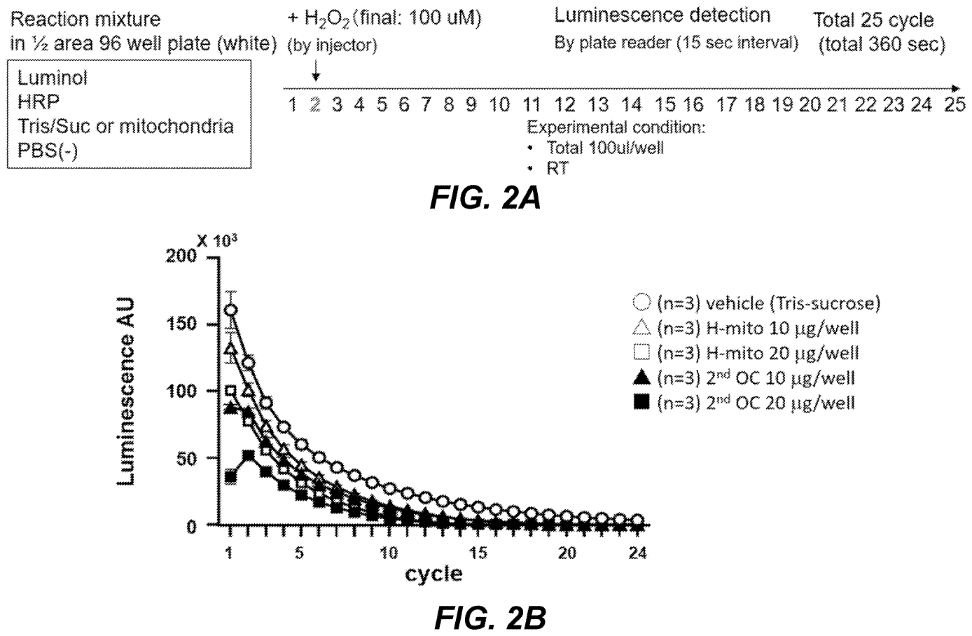

A luminol assay was employed for the detection of H₂O₂ by chemiluminescence (CL) (See React Oxyg Species (Apex). 2016 May; 1(3): 216-227. doi:10.20455/ros.2016.841, incorporated herein by reference). In the presence of horseradish peroxidase (HRP), luminol can react with hydrogen peroxide, ultimately leading to the formation of the excited state of 3-aminophthalate (3-APA*). 3-APA* decay to a lower energy level results in photon emission, which can be measured luminometrically as the chemiluminescence (CL) response. FIG. 2A depicts the experimental setup and FIG. 2B depicts results of a luminol assay comparing anti-ROS activity between second organelle complexes (2^{nd} OC) and homogenized mitochondria (H-mito). Calculated IC50 values are as follows: HeLa-derived second organelle complexes (2^{nd} OC), 153.4 μg/ml; first organelle complexes (1^{st} OC), 166.9 μg/ml; homogenized mitochondria, 167.4 μg/ml. These data demonstrate the anti-ROS effect of the various redox-modulating compositions provided herein.

### Example 3

### Anti-RNS Activity of Redox-Modulating Compositions

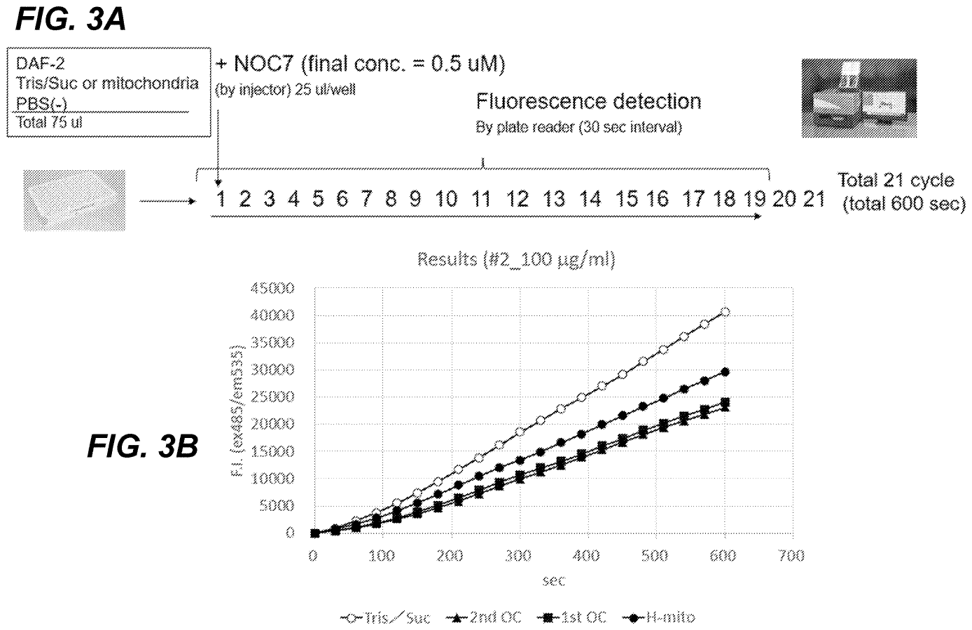

A nitric oxide detection assay was performed using the NOC7-DAF-2 system. NOC7 is employed as a nitric oxide donor and DAF-2 is employed as a nitric oxide detection agent. FIG. 3A depicts the experimental setup and FIG. 3B depicts data related to 0.5 μM NOC7 with DAF-2 (1 μM) assays. FIG. 3B depicts the results of a NOC7-DAF-2 assay employing 0.5M NOC7 and increasing doses of DAF-2. The anti-RNS activity was demonstrated with three different populations comprising mitochondria (second organelle complexes (2^{nd} OC), first organelle complexes (1^{st} OC), and homogenized mitochondria (H-mito)) relative to a control (Tris/Suc). 100 μg/mL (FIGS. 3B), 25 μg/mL (not shown), and 6.25 μg/mL (not shown) of the indicated populations These data demonstrate the dose-dependent anti-RNS effect of the various redox-modulating compositions provided herein.

### Example 4

### Cell-Based Assays of the Redox-Modulating Compositions

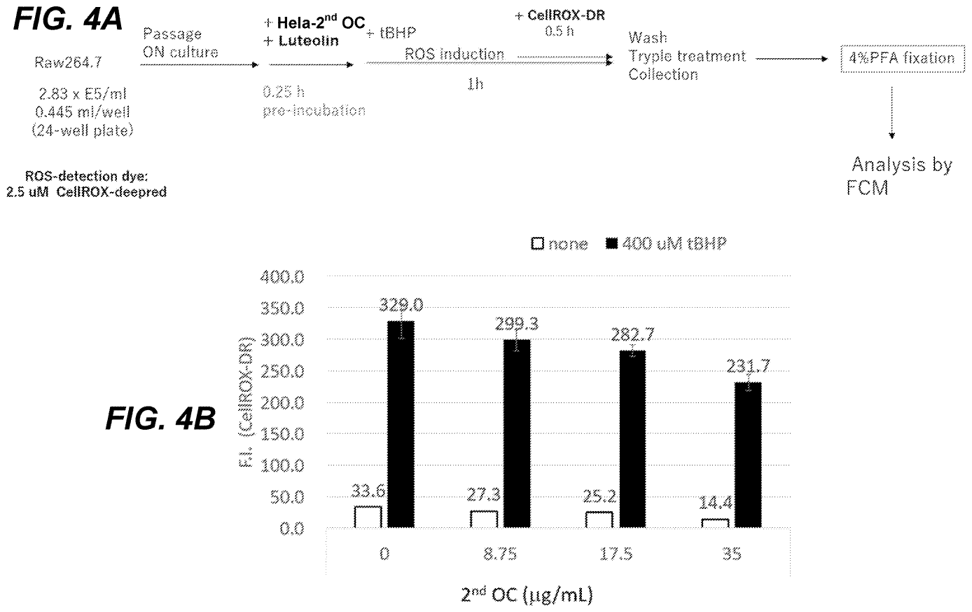

Cell-based assays were performed to examine the effect of the redox-modulating compositions described herein on cells. FIGS. 4A-4B depicts the experimental setup (FIG. 4A) and data (FIG. 4B) related to cell-based assays examining the in vitro redox-modulating activity of the compositions provided herein. FIG. 4B shows the results of the assay (performed in triplicate) with second organelle complexes (2^{nd} OC) derived from HeLa cells. Second organelle complexes (derived from HeLa cells) were found to suppress tBHP-induced ROS in dose-dependent manner.

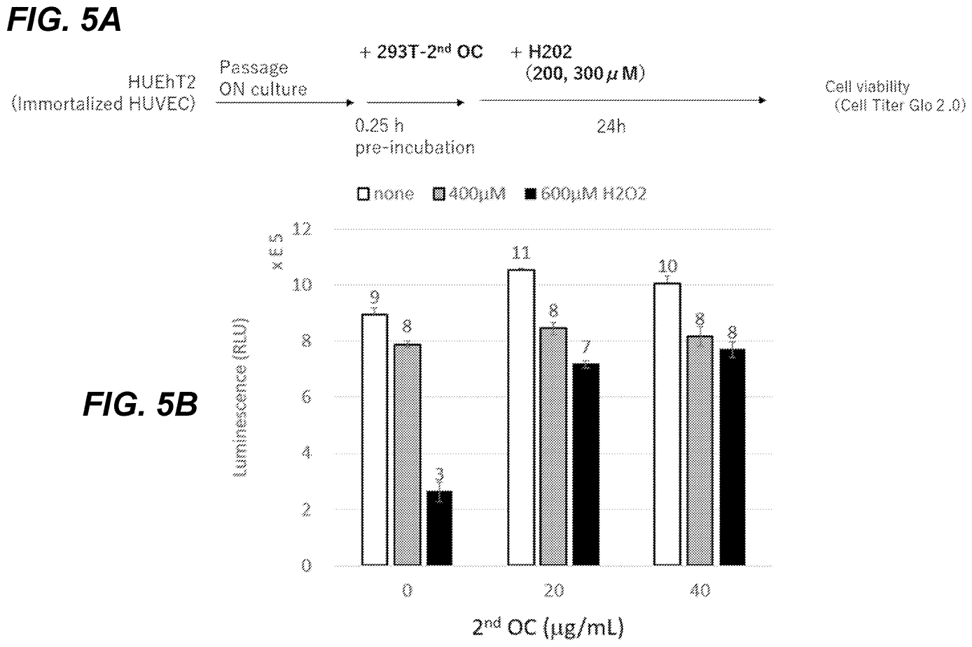

Next, CellTiter-Glo® 2.0 assays were conducted to examine the effect of the redox-modulating compositions described herein on HUEhT2 and Raw264.7 (data not shown) cells. The CellTiter-Glo® 2.0 Assay can determine the number of viable cells in culture by quantifying ATP, which can indicate the presence of metabolically active cells. Luminescence readout can be directly proportional to the number of viable cells in culture. FIGS. 5A-5B depict the experimental setup (FIG. 5A) and data (FIG. 5B) related to CellTiter-Glo® 2.0 assays examining the in vitro redox-modulating activity of the compositions provided herein. FIG. 5B depicts the results of a assay showing rescue of HUEhT2 cells by second organelle complexes (2^{nd} OC, derived from 293T cells). These data demonstrate the in vitro redox-modulating effect of the redox-modulating compositions provided herein.

### Example 5

### Cell Viability Assays

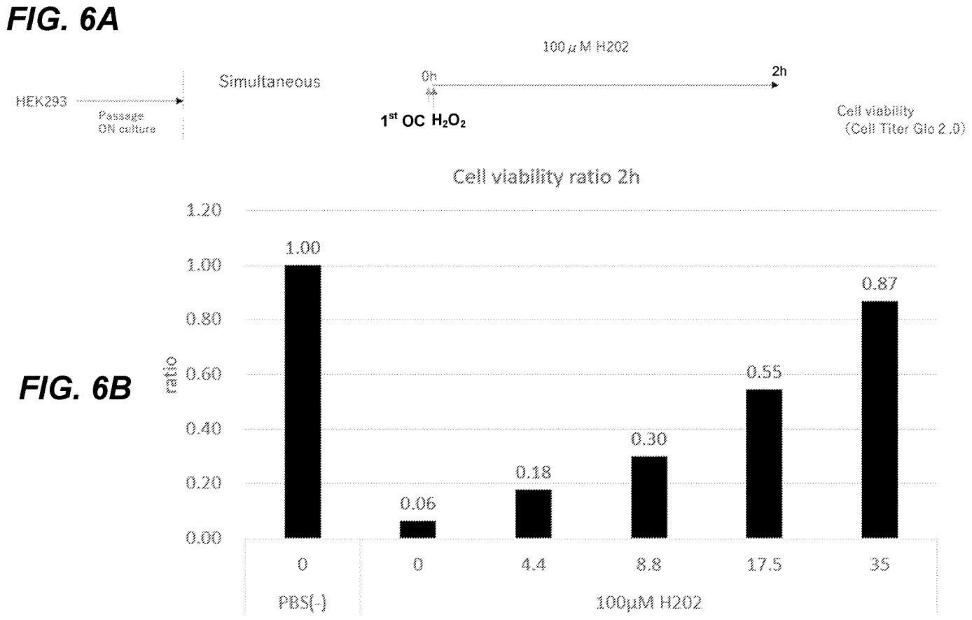

Cell viability assays were performed to evaluate the effect of the disclosed redox-modulating compositions. FIG. 6A depicts the experimental setup and FIG. 6B depicts data related to cell viability assays examining the redox-modulating compositions provided herein. The first organelle complexes (1^{st} OC) derived from HeLa cells improves cell viability ratios 2 hours and 4 hours (not shown) after contact with H₂O₂. First organelle complexes (derived from HeLa cells) were found to improve cell viability in a dose-dependent manner at both time points.

### Example 6

### NAD⁺/NADH Assays

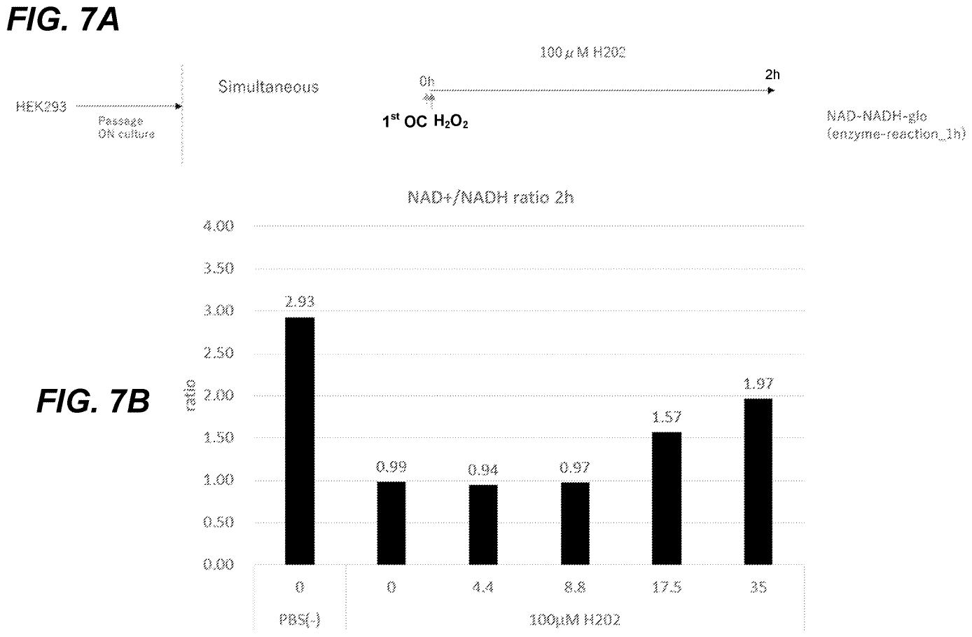

NAD⁺/NADH assays were performed to examine the impact of first organelle complexes on cells. FIG. 7A depicts the experimental setup and FIG. 7B depicts data related to NAD⁺/NADH assays examining the redox-modulating compositions provided herein. The first organelle complexes (1^{st} OC) derived from HeLa cells alters NAD⁺/NADH ratios 2 hours and 4 hours (not shown) after contact with H₂O₂. First organelle complexes (derived from HeLa cells) were found to increase NAD⁺/NADH ratios in a dose-dependent manner at both time points. In some embodiments, and without being bound by any particular theory, the redox-modulating compositions disclosed herein provide scavenging of reductive stress, and can reduce reductive stress by oxidizing NADH to NAD⁺, which can lead to antioxidant activity, ATP production capacity, and/or cell proliferative capacity.

### Example 7

### Comparison of First Organelle Complexes and Second Organelle Complexes

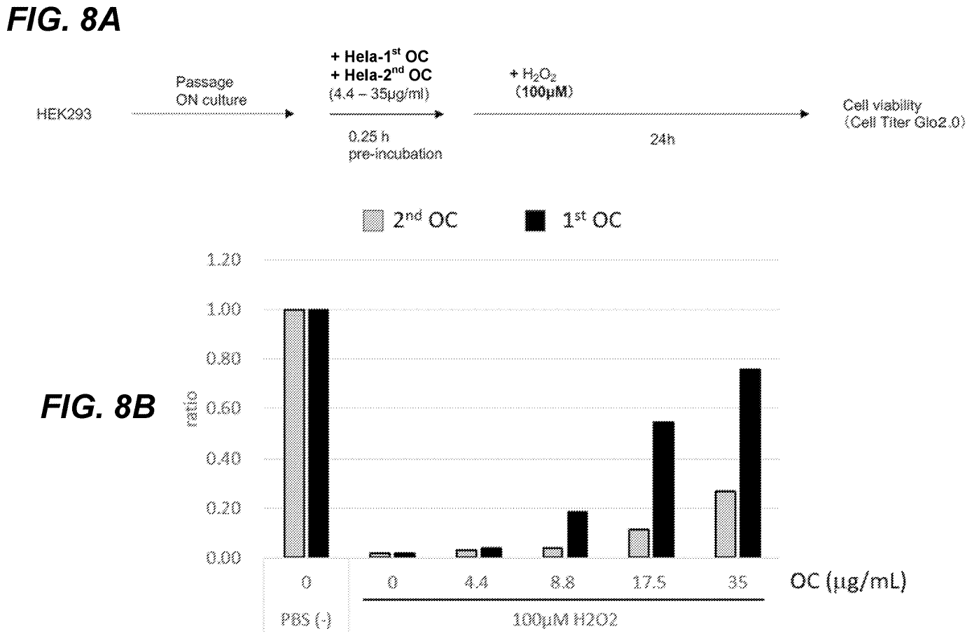

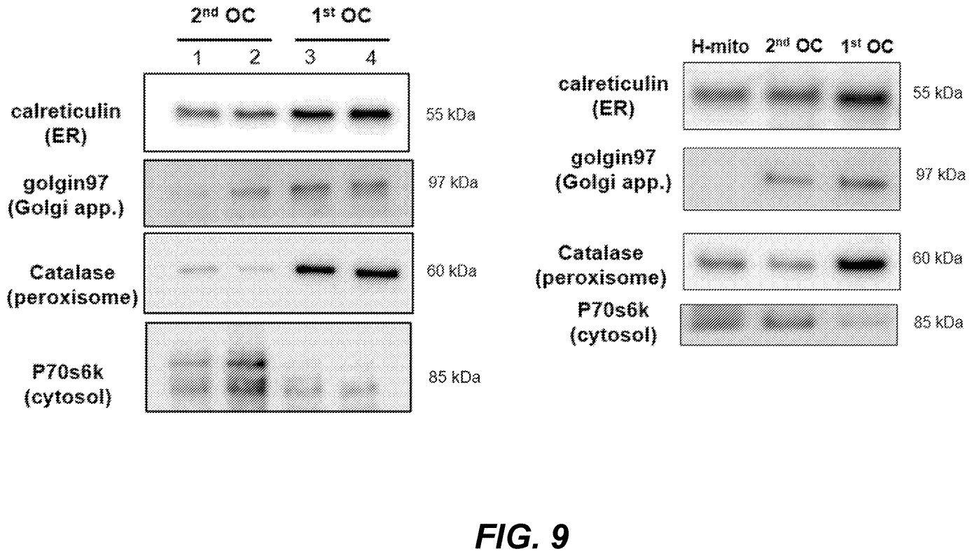

Cell-based assays were performed to compare the effect of first organelle complexes and second organelle complexes on viability. FIG. 8A depicts the experimental setup and FIG. 8B depicts data related to CellTiter-Glo® 2.0 assays comparing first organelle complexes (1^{st} OC) and second organelle complexes (2^{nd} OC) derived from HeLa cells. The results with first and second organelle complexes on cell viability (FIG. 8B) are shown ratio relative to PBS-treated control cells. First organelle complexes (derived from HeLa cells) were found to have (in a dose-dependent manner) stronger anti-reductive stress impact than second organelle complexes. The compositions of first organelle complexes and second organelle complexes were compared in view of these differences to elucidate the active components of the organelle complexes provided herein for anti-reducing stress indications. FIG. 9 depicts non-limiting exemplary data related to characterization of the organelle complexes populations provided herein. It shows intracellular structures/organelles western blot protein analysis of first organelle complexes (1^{st} OC) and second organelle complexes (2^{nd} OC) prepared using the methods disclosed herein from HEK293T cells.

### Example 8

### Elucidating Role of GSH and Catalase in the Anti-ROS Activity of First Organelle Complexes

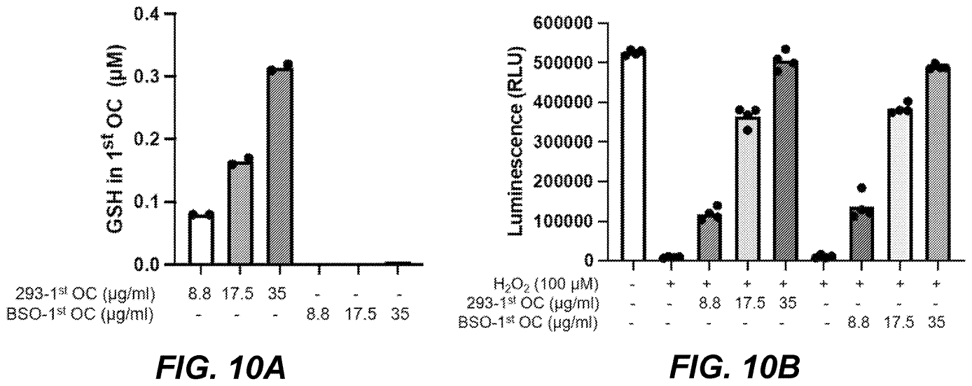

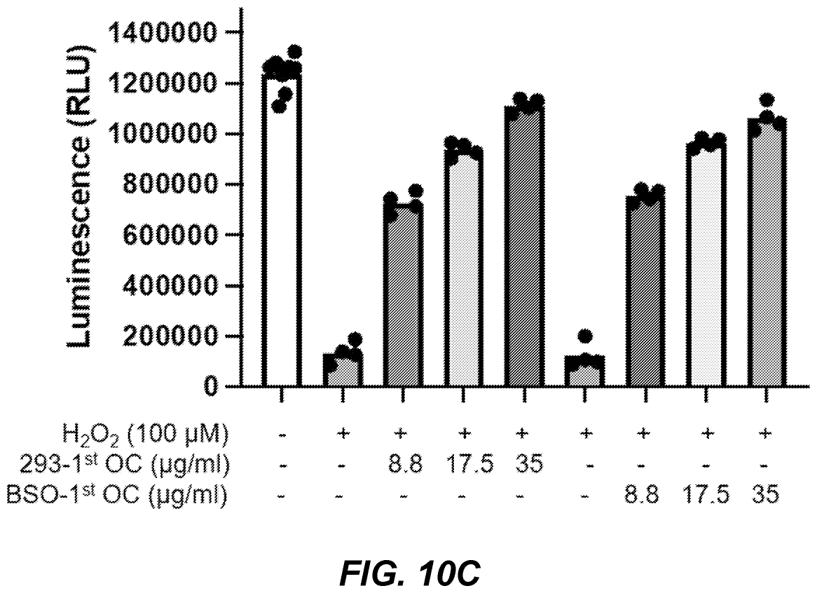

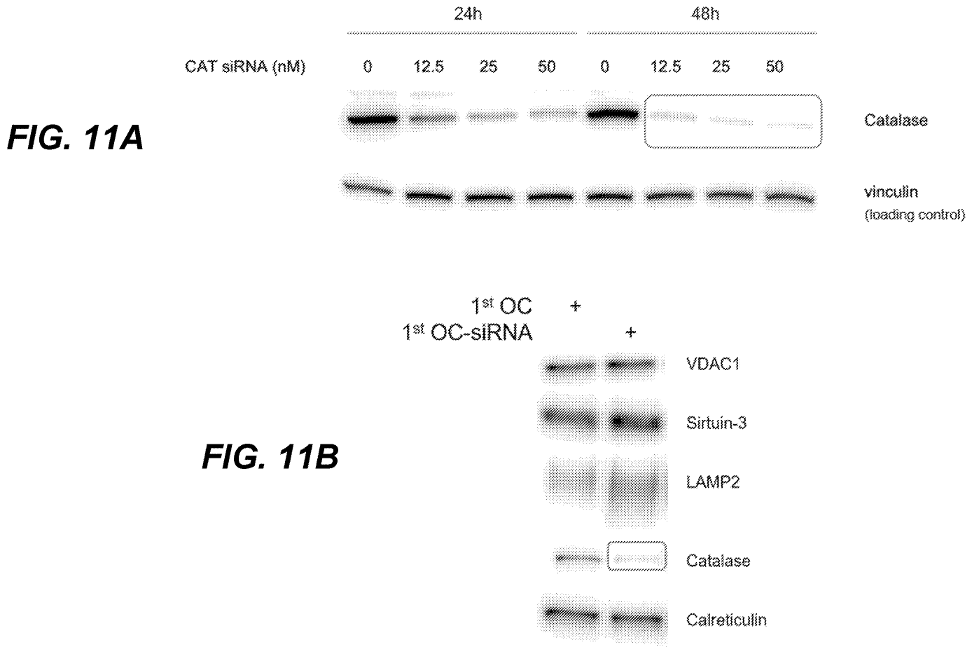

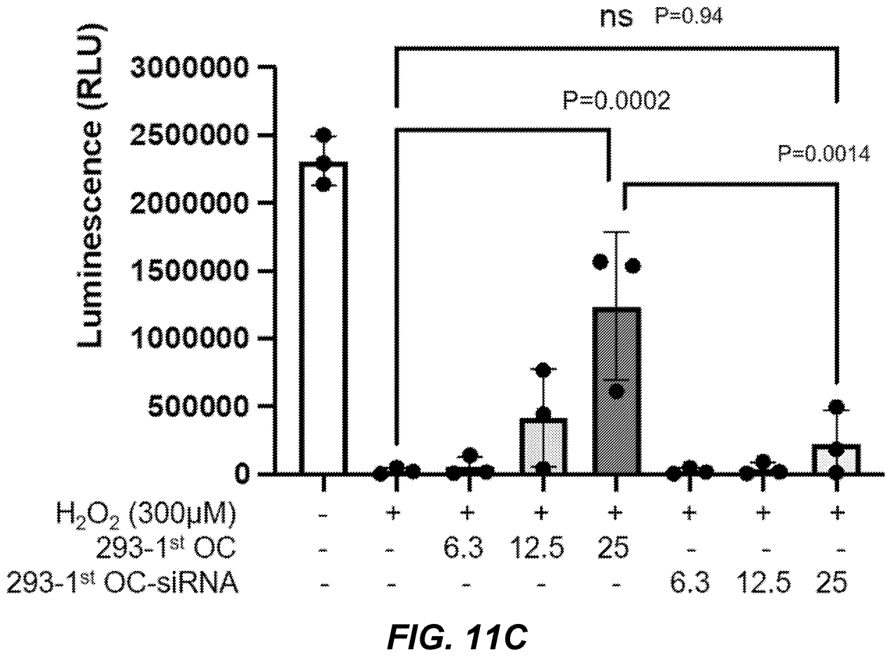

Cell-based assays were performed to examine the role of glutathione (GSH) and catalase in the anti-ROS activity of first organelle complexes. Buthionine sulfoximine (BSO) was employed to reduce GSH levels in first organelle complexes. FIG. 10A depicts GSH concentration in first organelle complexes derived from HEK293 cells that have (BSO-1^{st} OC) or have not (293-1^{st} OC) been contacted with BSO. An ON culture of HEK293 cells was passaged and incubated with H₂O₂ (100 μM) and either 293-1^{st} OC or BSO-1^{st} OC. The cell viability at 4 hours and 20 hours after H₂O₂ treatment were determined with CellTiter-Glo (CTG2.0). The results indicated that GSH in first organelle complexes is not an important factor in its anti-ROS activity (FIGS. 10B-10C). The ROS-scavenging activity of catalase-depleted first organelle complexes was next evaluated. HEK293 cells were transfected with catalase siRNA (h) (Santa cruz Biotechnology (SCB); sc-45330; lot #B2423) using common transfection reagent for either 24 hours or 48 hours, and the expression level of catalase in the total cell lysates were evaluated by Western blotting analysis (FIG. 11A). FIG. 11B depicts catalase levels in untreated first organelle complexes (1^{st} OC) or catalase-depleted first organelle complexes (1^{st} OC-siRNA). Having confirmed successful knockdown of catalase in first organelle complexes, the ROS scavenging activity of catalase-depleted first organelle complexes (1^{st} OC-siRNA) was evaluated. HEK293 cells were pretreated with either first organelle complexes (293-1^{st} OC) or catalase-depleted first organelle complexes (293-1^{st} OC-siRNA) for 6 hours, followed by 18 hours of H₂O₂ (300 μM) treatment. The cell viability was measured with CellTiter-Glo (CTG2.0). It was found that knockdown of catalase in first organelle complexes reduces its ROS-scavenging activity, with statistically significance (P<0.005) (FIG. 11C). These results provided in this Example show that GSH depletion in first organelle complexes by BSO does not affect its anti-ROS activity, while the presence of catalase in organelle complexes (e.g., first organelle complexes, second organelle complexes) is important factor for its ROS-scavenging activity.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of reducing or preventing oxidative stress and/or reductive stress in a redox-sensitive composition, comprising:
contacting a redox-sensitive composition with an effective amount of a redox-modulating composition, thereby reducing or preventing oxidative stress and/or reductive stress in the redox-sensitive composition,
wherein the redox-modulating composition comprises isolated organelle complexes, wherein the organelle complexes comprise mitochondria and one or more of endoplasmic reticulum, peroxisomes, lysosomes, and Golgi apparatus.

2. A method of reducing levels of reactive oxygen species (ROS) and/or reactive nitrogen species (RNS) in a redox-sensitive composition, comprising:
contacting a redox-sensitive composition with an effective amount of a redox-modulating composition, thereby reducing levels of ROS and/or RNS in the redox-sensitive composition,
wherein the redox-modulating composition comprises isolated organelle complexes, wherein the organelle complexes comprise mitochondria and one or more of endoplasmic reticulum, peroxisomes, lysosomes, and Golgi apparatus.

3. The method of any one of claims 1-2, wherein the contacting step comprises incubating a mixture of the redox-sensitive composition and the redox-modulating composition for less than about 30 seconds.

4. The method of any one of claims 1-3, wherein the effective amount comprises at least about 5 ug/mL to about 5 mg/mL of the redox-modulating composition.

5. The method of any one of claims 1-4, wherein the redox-sensitive composition is experiencing oxidative stress and/or reductive stress or is at risk of experiencing oxidative stress and/or reductive stress.

6. The method of any one of claims 1-5, wherein the redox-sensitive composition comprises one or more cells, optionally the contacting step comprises introducing the redox-modulating composition into the one or more cells, further optionally the one or more cells are cells of a subject, further optionally the one or more cells is undergoing or at risk of undergoing hypoxia.

7. The method of any one of claims 1-6, wherein the contacting is performed ex vivo, in vitro, or in vivo.

8. The method of any one of claims 1-7, wherein the redox-sensitive composition is an oxidant-sensitive composition, a reductant-sensitive composition, or a combination thereof.

9. The method of any one of claims 1-8, wherein the redox-sensitive composition comprises a biological sample.

10. The method of any one of claims 1-9, wherein the biological sample is selected from the group consisting of a soil sample, an air sample, an environmental sample, a cell culture sample, a bone marrow sample, a rainfall sample, a fallout sample, a space sample, an extraterrestrial sample, a sewage sample, a ground water sample, an abrasion sample, an archaeological sample, a food sample, a blood sample, a serum sample, a plasma sample, a urine sample, a stool sample, a semen sample, a lymphatic fluid sample, a cerebrospinal fluid sample, a naspharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a nail sample, a hair sample, a skin sample, a cancer sample, a tumour sample, a tissue sample, a cell sample, a cell lysate sample, a virus culture sample, a forensic sample, an infection sample, a nosocomial infection sample, a production sample, a drug preparation sample, a biological molecule production sample, a protein preparation sample, a lipid preparation sample, a carbohydrate preparation sample, a solution of a nucleotide, a solution of polynucleotide, a solution of a nucleic acid, a solution of a peptide, a solution of a polypeptide, a solution of an amino acid, a solution of a protein, a solution of a synthetic polymer, a solution of a biochemical composition, a solution of an organic chemical composition, a solution of an inorganic chemical composition, a solution of a lipid, a solution of a carbohydrate, a solution of a combinatory chemistry product, a solution of a drug candidate molecule, a solution of a drug molecule, a solution of a drug metabolite, a suspension of a cell, a suspension of a virus, a suspension of a microorganism, a suspension of a metal, a suspension of metal alloy, a solution of a metal ion, and any combination thereof.

11. A method of reducing or preventing oxidative stress and/or reductive stress in a subject, comprising:
administering to the subject an effective amount of a redox-modulating composition, thereby reducing or preventing oxidative stress and/or reductive stress in the subject,
wherein the redox-modulating composition comprises isolated organelle complexes, wherein the organelle complexes comprise mitochondria and one or more of endoplasmic reticulum, peroxisomes, lysosomes, and Golgi apparatus.

12. A method of treating or preventing a redox disease or disorder in a subject, comprising:
administering to the subject an effective amount of a redox-modulating composition, thereby treating or preventing a redox disease or disorder in a subject,
wherein the redox-modulating composition comprises isolated organelle complexes, wherein the organelle complexes comprise mitochondria and one or more of endoplasmic reticulum, peroxisomes, lysosomes, and Golgi apparatus.

13. The method of any one of claims 1-12, wherein the redox-modulating composition comprises homogenized mitochondria, first organelle complexes, and/or second organelle complexes,
wherein the first organelle complexes and second organelle complexes comprise mitochondria and one or more of endoplasmic reticulum, peroxisomes, lysosomes, and Golgi apparatus,
wherein the first organelle complexes and second organelle complexes are depleted of cytosolic macromolecules,
wherein first organelle complexes are derived from (i) frozen cells; (ii) floating cells; and/or (iii) cells contacted with a surfactant at a concentration at or above the critical micellar concentration (CMC) for the surfactant, and
wherein second organelle complexes are derived from (i) adherent cells; and/or (ii) cells contacted with a surfactant at a concentration below the critical micellar concentration (CMC) for the surfactant,
optionally the first organelle complexes and second organelle complexes are derived from cells treated with a mitochondria-activating agent,
further optionally the homogenized mitochondria, first organelle complexes, and/or second organelle complexes are encapsulated in lipid membrane-based vesicles.

14. The method of any one of claims 1-13, wherein the effective amount comprises at least about 1 ug to about 1 mg of the redox-modulating composition.

15. The method of any one of claims 1-14, wherein the redox-modulating composition does not comprise intact cells.

16. The method of any one of claims 1-15, wherein the oxidative stress and/or reductive stress comprises elevated levels of ROS, RNS, and/or free radicals.

17. The method of any one of claims 1-16, wherein the oxidative stress and/or reductive stress comprises altered cell functions.

18. The method of any one of claims 1-17, wherein the oxidative stress and/or reductive stress is associated with the redox disease or disorder.

19. The method of any one of claims 1-18, wherein the redox-modulating composition reduces levels of one or more ROS and/or one or more RNS in the subject or the redox-sensitive composition by at least about 5%.

20. The method of any one of claims 1-19, wherein the redox-modulating composition reduces level of oxidative cell stress and thereby recover or restore the cell functions.

21. The method of any one of claims 1-20, wherein the redox-modulating composition has a ROS scavenging activity and/or RNS scavenging activity.

22. The method of any one of claims 1-21, wherein the redox-modulating composition reduces or prevents ROS generation and/or RNS generation in the subject.

23. The method of any one of claims 1-22, wherein the redox-modulating composition has superoxide dismutase activity, catalase activity, peroxidase activity, or any combination thereof.

24. The method of any one of claims 1-23, wherein the redox-modulating composition increases and/or decreases one or more of the following ratios in the subject or the redox-sensitive composition: oxidized to reduced forms of nicotinamide adenine dinucleotide (NAD⁺/NADH), oxidized to reduced forms of nicotinamide adenine dinucleotide phosphate (NADP⁺/NADPH), oxidized to reduced forms of glutathione (GSSG/GSH), and oxidized to reduced forms of thioredoxin (TrxSS/TrxSH₂).

25. The method of any one of claims 1-24, wherein the reactive nitrogen species comprise nitric oxide (NO), nitrogen dioxide radical (·NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) or dinitrogen trioxide (N₂O₃), or any combination thereof.

26. The method of any one of claims 1-25, wherein the reactive oxygen species comprise superoxide (O₂·⁻), hydroperoxy (HO·₂), hydrogen peroxide (H₂O₂), peroxynitrite (ONOO—), hypochlorous acid (HOCl), hypobromous acid (HOBr), hydroxyl radical (HO·), peroxy radical (ROO·), alkoxy radical (RO·), singlet oxygen (¹O₂), lipid peroxides, lipid peroxyradicals or lipid alkoxyl radicals, or any combination thereof.

27. The method of any one of claims 1-26, wherein the redox-modulating composition has free radical scavenging activity and/or inhibits free radical formation.

28. The method of any one of claims 1-27, wherein the subject is experiencing oxidative stress and/or reductive stress or is at risk of experiencing oxidative stress and/or reductive stress, optionally caused by ischemic-reperfusion injury or the redox disease or disorder.

29. The method of any one of claims 1-28, wherein the method comprises identifying a subject experiencing oxidative stress and/or reductive stress or at risk of experiencing oxidative stress and/or reductive stress.

30. The method of any one of claims 1-29, comprising:
measuring the levels of RNS in the subject or the redox-sensitive composition with a probe that detects reactive nitrogen species; and/or
measuring the levels of ROS in the subject or the redox-sensitive composition with a probe that detects reactive oxygen species.

31. The method of any one of claims 1-30, wherein the probe that detects reactive nitrogen species is diaminonaphthalene, a diaminofluorescein, a diaminorhodamine, a diaminocyanine, an NiSPY, dichlorodiaminocalcein, 1,2-diaminoanthraquinone, or DAMBO-P^{H}.

32. The method of any one of claims 1-31, wherein the probe that detects reactive oxygen species is 2′,7′-dichloro-fluorescein diacetate, dihydrorhodamine 123, 3′-(p-aminophenyl) fluorescein (API), 3′-(p-hydroxyphenyl) fluorescein (HPF), aminophenoxycalcein (APC), mitoAR, mitoHR, DPAX, DMAX, a hydrocyanine, or dihydroethidium.

33. The method of any one of claims 1-32, wherein the ROS are derived from neutrophils and/or xanthine oxidase (XO).

34. The method of any one of claims 1-33, wherein the subject has or is suspected of having a disease or condition in which ROS is implicated selected from the group consisting of atherosclerosis, heart disease, heart failure, hypertension, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, stroke, an ischemia-reperfusion injury, an ischemia-reperfusion injury, chronic inflammation, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack, UV damage to the skin, organ transplantation, surgery, and any combination thereof.

35. The method of any one of claims 1-34, wherein administering the redox-modulating composition to the subject treats, reduces, or prevents ischemia-reperfusion injury of the subject.

36. The method of any one of claims 1-35, wherein the ischemic-reperfusion injury is caused by mitochondrial dysfunction, hypoxic injury, HMGB1 release or necrotic cell death.

37. The method of any one of claims 1-36, wherein the redox-modulating composition suppresses ischemic-reperfusion injury, mitochondrial dysfunction, hypoxic injury, necrotic cell death, or any combination thereof.

38. The method of any one of claims 1-37, wherein the redox disease or disorder comprises ROS-mediated and/or RNS-mediated oxidative damage to one or more tissues of the subject.

39. The method of any one of claims 1-38, wherein the redox disease or disorder is selected from the group consisting of: a mitochondrial disorder; an inherited mitochondrial disease; Alpers Disease; Barth syndrome; a Beta-oxidation Defect; Carnitine-Acyl-Carnitine Deficiency; Carnitine Deficiency; a Creatine Deficiency Syndrome; Co-Enzyme Q10 Deficiency; Complex I Deficiency; Complex II Deficiency; Complex III Deficiency; Complex IV Deficiency; Complex V Deficiency; COX Deficiency; chronic progressive external ophthalmoplegia (CPEO); CPT I Deficiency; CPT II deficiency; Friedreich's Ataxia (FA); Glutaric Aciduria Type II; KeaRNS-Sayre Syndrome (KSS); Lactic Acidosis; Long-Chain Acyl-CoA Dehydrongenase Deficiency (LCAD); LCHAD; Leigh Syndrome; Leigh-like Syndrome; Leber's Hereditary Optic Neuropathy (LHON); Lethal Infantile Cardiomyopathy (LIC); Luft Disease; Multiple Acyl-CoA Dehydrogenase Deficiency (MAD); Medium-Chain Acyl-CoA Dehydrongenase Deficiency (MCAD); Mitochondrial Myopathy, Encephalopathy, Lactacidosis, Stroke (MELAS); Myoclonic Epilepsy with Ragged Red Fibers (MERRF); Mitochondrial Recessive Ataxia Syndrome (MIRAS); Mitochondrial Cytopathy, Mitochondrial DNA Depletion; Mitochondrial Encephalopathy; Mitochondrial Myopathy; Myoneurogastointestinal Disorder and Encephalopathy (MNGIE); Neuropathy, Ataxia, and Retinitis Pigmentosa (NARP); Pearson Syndrome; Pyruvate Carboxylase Deficiency; Pyruvate Dehydrogenase Deficiency; a Respiratory Chain Disorder; Short-Chain Acyl-CoA Dehydrogenase Deficiency (SCAD); SCHAD; Very Long-Chain Acyl-CoA Dehydrongenase Deficiency (VLCAD); a myopathy; cardiomyopathy; encephalomyopathy; a neurodegenerative disease; Parkinson's disease; Alzheimer's disease; amyotrophic lateral sclerosis (ALS); a motor neuron disease; a neurological disease; epilepsy; an age-associated disease; macular degeneration; diabetes; metabolic syndrome; brain cancer; a genetic disease; Huntington's Disease; a mood disorder; schizophrenia; bipolar disorder; a pervasive developmental disorder; autistic disorder; Asperger's syndrome; childhood disintegrative disorder (CDD); Rett's disorder; PDD-not otherwise specified (PDD-NOS); a cerebrovascular accident; stroke; a vision impairment; optic neuropathy; dominant inherited juvenile optic atrophy; optic neuropathy caused by a toxic agent; glaucoma; Stargardt's macular dystrophy; diabetic retinopathy; diabetic maculopathy; retinopathy of prematurity; ischemic reperfusion related retinal injury; oxygen poisoning; a haemoglobionopathy; thalassemia; sickle cell anemia; seizures; ischemia; renal tubular acidosis; attention deficit/hyperactivity disorder (ADHD); a neurodegenerative disorder resulting in hearing or balance impairment; Dominant Optic Atrophy (DOA); Maternally inherited diabetes and deafness (MIDD); chronic fatigue; contrast-induced kidney damage; contrast-induced retinopathy damage; Abetalipoproteinemia; retinitis pigmentosum; Wolfram's disease; Tourette syndrome; cobalamin c defect; methylmalonic aciduria; glioblastoma; Down's syndrome; acute tubular necrosis; a muscular dystrophy; a leukodystrophy; Progressive Supranuclear Palsy; spinal muscular atrophy; hearing loss; noise induced hearing loss; traumatic brain injury; Juvenile Huntington's Disease; Multiple Sclerosis; NGLY1; Multisystem atrophy; Adrenoleukodystrophy; Adrenomyeloneuropathy; and any combination thereof.

40. The method of any one of claims 1-39, wherein the redox-modulating composition improves one or more energy biomarkers in the subject or the redox-sensitive composition selected from the group consisting of: lactic acid (lactate) levels; pyruvic acid (pyruvate) levels; lactate/pyruvate ratios; total, reduced or oxidized glutathione levels, or reduced/oxidized glutathione ratio; total, reduced or oxidized cysteine levels, or reduced/oxidized cysteine ratio; phosphocreatine levels, NADH (NADH+H⁺) levels; NADPH (NADPH+H⁺) levels; NAD levels; NADP levels; ATP levels; reduced coenzyme Q (CoQred) levels; oxidized coenzyme Q (CoQox) levels; total coenzyme Q (CoQtot) levels; oxidized cytochrome C levels; reduced cytochrome C levels; oxidized cytochrome C/reduced cytochrome C ratio; acetoacetate levels, b-hydroxy butyrate levels, acetoacetate/b-hydroxy butyrate ratio, 8-hydroxy-2′-deoxyguanosine (8-OHdG) levels; levels of reactive oxygen species; levels of oxygen consumption (V02); levels of carbon dioxide output (VC02); respiratory quotient (VC02/V02); exercise tolerance; anaerobic threshold; and any combination thereof.

41. The method of any one of claims 1-40, wherein the administering comprises intravenous administration, intra-arterial administration, intra-tracheal administration, subcutaneous, administration intramuscular administration, inhalation, intrapulmonary administration, and/or intraocular administration.
